(19) United States
(12) Patent Application Publication
(10) Pub. No.: US 2026/0369007 A1
(43) Pub. Date: Nov. 19, 2026

(54) SYSTEMS AND METHODS FOR 3-DIMENSIONAL ANATOMICAL RECONSTRUCTION USING MONOSCOPIC PHOTOGRAMMETRY

(71) Applicant: Dignity Health, San Francisco, CA (US)

(72) Inventors: Sahin Hanalioglu, San Francisco, CA (US); Giancarlo Mignucci, San Francisco, CA (US); Mark Preul, San Francisco, CA (US); Nicolas I. Gonzalez Romo, San Francisco, CA (US)

(21) Appl. No.: 19/142,748

(22) PCT Filed: Dec. 26, 2023
(86) PCT No.: PCT/US2023/085928

§ 371 Date: Jun. 24, 2025

Related U.S. Application Data
(60) Provisional application No. 63/477,185, filed on Dec. 24, 2022.

(51) Int. Cl. G06T 17/20; G06T 7/55; G06T 19/00; G09B 5/02; G16H 40/67

(52) U.S. Cl. CPC G06T 17/20; G06T 7/55; G06T 19/006; G09B 5/02; G16H 40/67; G06T 2207/10028; G06T 2207/20081; G06T 2207/20084; G06T 2207/30004; G06T 2210/41; G06T 2210/56

(57) ABSTRACT

A system applies a monoscopic photogrammetry process that transforms 2D high resolution anatomical images into volumetric surface reconstructions. The system includes a neural network capable of monoscopic depth estimation to generate a set of depth estimation data from a 2D anatomical image. The system then transforms the depth map into a navigable 3D model for display within an extended reality environment.

### CROSS-REFERENCE TO RELATED APPLICATIONS

This is a PCT application that claims benefit to U.S. Provisional Application Ser. No. 63/477,185 filed 24 Dec. 2022, which is incorporated by reference in its entirety.

### FIELD

The present disclosure generally relates to anatomical modeling, and in particular, to a system and associated methods for three-dimensional anatomical reconstruction using a monoscopic photogrammetry technique.

### BACKGROUND

Immersive anatomical environments offer an alternative when anatomical laboratory access is limited, but current three-dimensional (3D) renderings are not able to simulate the anatomical detail and surgical perspectives needed for microsurgical education.

It is with these observations in mind, among others, that various aspects of the present disclosure were conceived and developed.

### BRIEF DESCRIPTION OF THE DRAWINGS

FIG. 1A is a simplified block diagram showing a system for generating 3D anatomical models from 2-D images using monoscopic photogrammetry;

FIGS. 1B-1E are a series of images showing sequential generation of a 3D anatomical model from a 2-D image by the system of FIG. 1A;

FIG. 2 is a simplified diagram showing an exemplary computing system for implementation of the system of FIG. 1A;

FIG. 3 is a simplified diagram showing an example neural network architecture model for implementation of the system of FIG. 1A;

FIG. 4 is a simplified diagram showing a training sequence for training the neural network

FIGS. 5A-5C are a series of images showing an example user interface of the system of FIG. 1A and sequential generation of a 3D anatomical model;

FIGS. 6A-6D are a series of images showing an extended reality environment for display of a 3D anatomical model of the system of FIG. 1A;

FIGS. 7A-7D are a series of images showing QR codes for the extended reality environment of the system of FIG. 1A;

FIGS. 8A-8I are a series of images showing photographs, depth maps and histograms of a Transsylvian exposure at varying magnification;

FIGS. 9A-9I are a series of images showing photographs, depth maps and histograms of a mastoidectomy dissection at varying magnification according to the system of FIG. 1A;

FIGS. 10A-10D are a series of images showing high-magnification photographs and their corresponding depth maps, with labeled anatomical ROIs according to the system of FIG. 1A; and

FIGS. 11A-11D are a series of images showing a 3D reconstruction procedure according to the system of FIG. 1A.

Corresponding reference characters indicate corresponding elements among the view of the drawings. The headings used in the figures do not limit the scope of the claims.

### DETAILED DESCRIPTION

### 1. Introduction

Advances in artificial intelligence-based object detection and three-dimensional (3D) reconstruction have become fundamental for modern-day applications, such as self-driving vehicles, military aircraft and vehicle training, and face recognition software. Ongoing research has expanded the applications of this technology in neurosurgery, where new strategies have led to an evolution in the neurosurgical education paradigm. Trainees can now supplement their anatomical exposure with online platforms, and microsurgical libraries that contain high-resolution photographs of dissected anatomy.

For microsurgical anatomy, machine learning could offer new insights into surgical exposure analysis through a computer science perspective that augments the learning of anatomy with novel 3D reconstruction techniques. Traditional photogrammetry allows conversion of still pictures into 3D objects through the acquisition of multiple images from different angles of the subject to create a point cloud for reconstruction. Photogrammetry, traditionally performed with multiple images of an object obtained at different angles, is one such computer-aided image reconstruction procedure in which 3D information is extracted from photographs.

### 2. System Overview

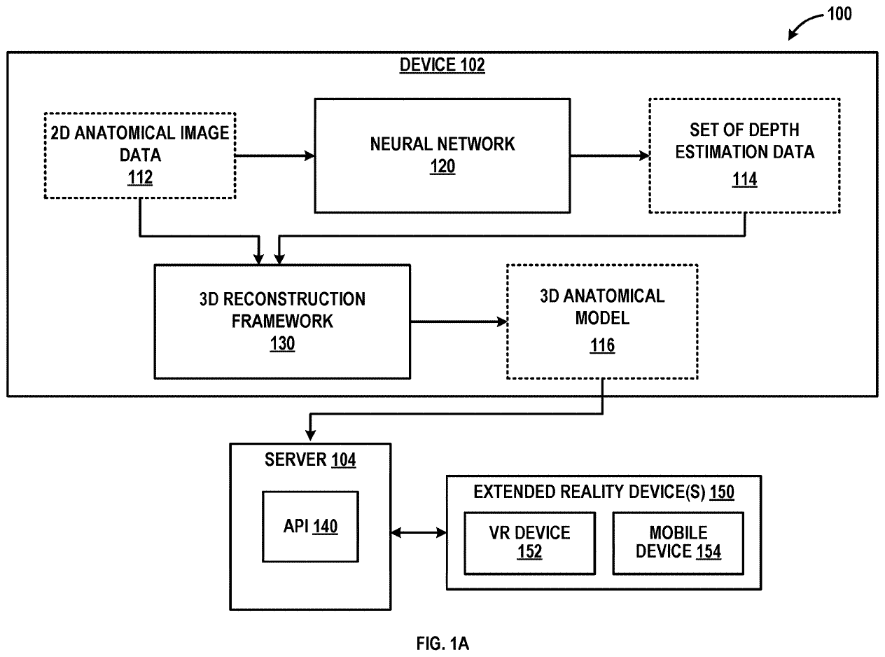

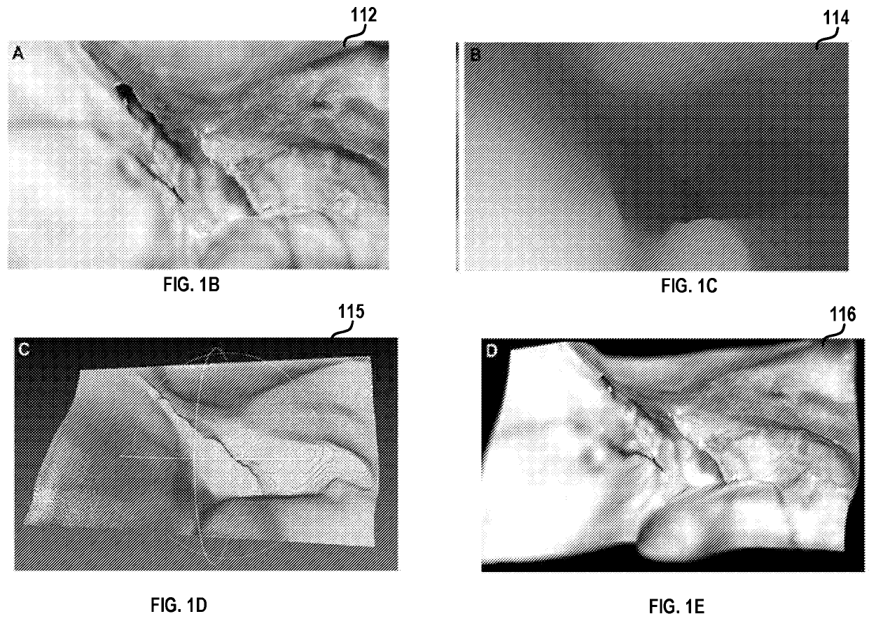

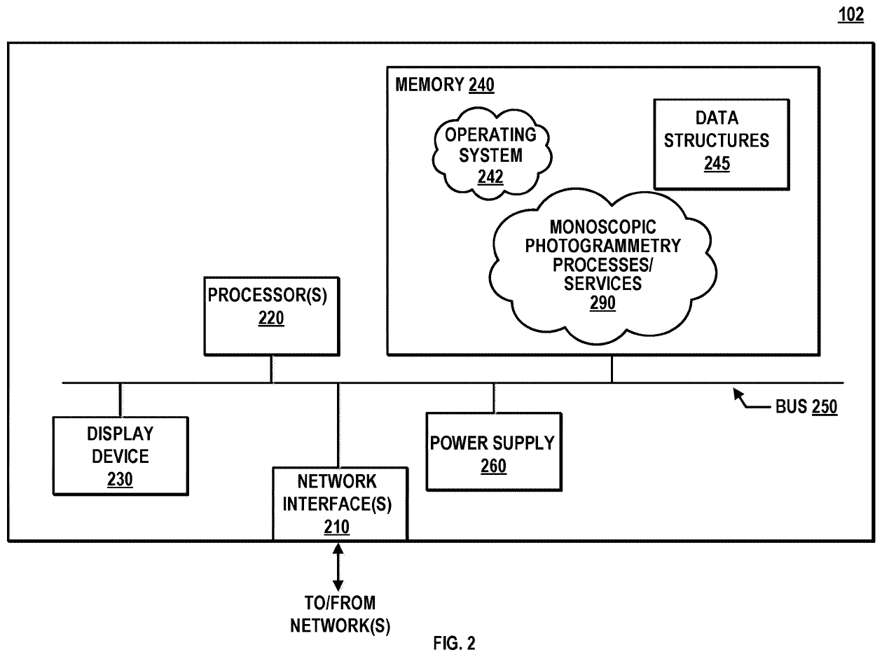

The present disclosure outlines a system and associated methods for computer-implemented 3D anatomical model reconstruction (hereinafter, system 100) that applies machine learning principles to transform high-resolution photographs into immersive anatomical models, offering a convenient and efficient resource for neurosurgical education. The system 100 applies a 3D reconstruction process (e.g., monoscopic photogrammetry) that transforms two-dimensional (2D) high-resolution anatomical input images into volumetric surface reconstructions. First, the system 100 applies an anatomical image as input to a neural network capable of monoscopic depth estimation to create a depth map. The system 100 can then apply a 3D reconstruction process to the depth map to transform the anatomical image into navigable 3D models. In one implementation, with reference to FIG. 1A, the system 100 includes a device 102 (e.g., a computing device) that implements a neural network 120 operable for monoscopic depth estimation that receives a set of 2D anatomical image data 112 and outputs a set of depth estimation data 114 (e.g., a depth map) for the set of 2D anatomical image data 112 using a monoscopic depth estimation process. The system 100 further implements a 3D reconstruction framework 130 that transforms the set of depth estimation data 114 into a 3D anatomical model 116. In a further aspect, the system 100 can include a server 104 operable for communication with an extended reality display device 150 such as a virtual reality display device 152 or a mobile device 154 (e.g., a smartphone), where the server 104 hosts an application programming interface (API) 140 for display of and interaction with the 3D anatomical model 116 at the extended reality display device 150.

This technology allows accurate spatial representation of micro-anatomical structures that are usually not well represented in digital renderings or DICOM reconstructions. The system 100 only requires one high resolution image, instead of multiple images as in traditional photogrammetry. Further, the system 100 performs a spatial correlation analysis using neuronavigation to ensure accuracy of depth estimations.

FIGS. 1B-1E illustrate sequential generation of a 3D anatomical model 116 from a set of 2D anatomical image data 112 by the system 100. FIG. 1B shows a set of 2D anatomical image data 112, which is provided as input to the neural network 120. FIG. 1C shows a resultant set of depth estimation data 114 which is an output of the neural network 120 represented visually using a gradient (e.g., in which “lighter” pixels represent “closer” portions of the 2D anatomical image data 112 and “darker” pixels represent “farther” portions of the 2D anatomical image data 112). Note that while FIG. 1C shows one example representation of the set of depth estimation data 114 as an image with a color value of each respective pixel corresponding to a “depth” value, the set of depth estimation data 114 can be represented within a comma-separated-variable (.csv) file, a bitmap (.bmp) file, or another suitable file format where each respective pixel in the 2D anatomical image data 112 is associated with a “depth” value.

FIGS. 1D and 1E show generation of a 3D anatomical model 116 at the 3D reconstruction framework 130 of the system 100 using the set of depth estimation data 114. In particular, FIG. 1D shows a 3D mesh 115 of the 3D anatomical model 116 generated using the 2D anatomical image data 112 and the set of depth estimation data 114. Finally, FIG. 1E shows a final render of the 3D anatomical model 116 including the 3D mesh 115 and also including color information for each pixel within the 2D anatomical image data 112. The 3D anatomical model 116 can be viewed at a display device within an extended reality environment and/or at a standard computing device.

A method implemented by system 100 herein includes: accessing the set of 2D anatomical image data 112 and generating the set of depth estimation data 114 based on the set of 2D anatomical image data 112 by a monoscopic photogrammetry procedure.

Generating the set of depth estimation data 114 can be performed by the neural network 120, the neural network 120 accepting the set of 2D anatomical image data 112 as input and being trained to output the set of depth estimation data 114 that correlates with the set of 2D anatomical image data 112. In some examples, the neural network 120 can compute an inverse depth value (e.g., depth or “Z” values) for each 2D coordinate pair of a plurality of 2D coordinate pairs (e.g., horizontal and vertical or “X and Y” values) of the set of 2D anatomical image data 112, and can generate a 3D point cloud of the set of depth estimation data 114 by combination of each 2D coordinate pair and the inverse depth value for the plurality of 2D coordinate pairs of the set of 2D anatomical image data 112.

The method can further include generating the 3D mesh 115 of the 3D anatomical model 116 using the set of depth estimation data 114. This step can encompass converting the 3D point cloud (represented by coordinate pairs with X, Y, and Z values) to the 3D mesh 115 of the 3D anatomical model 116. Finally, the method can include generating a final rendering of the 3D anatomical model 116 using the set of 2D anatomical image data 112 and the 3D mesh 115,

In a further aspect, the method can include communicating the final rendering of the 3D anatomical model 116 to the server 104 hosting the API 140 operable for communication with the extended reality device 150. When the extended reality device 150 is the virtual reality display device 152, the API 140 provides instructions executable by a processor associated with the virtual reality display device 152 to display the final rendering of the 3D anatomical model 116 within a virtual reality environment. When the extended reality device is the mobile device 154, the API 140 provides instructions executable by a processor of the mobile device 154 to display the final rendering of the 3D anatomical model 116 within an augmented-reality environment, These instructions can include superimposing the final rendering of the 3D anatomical model 116 over a live feed captured by a camera of the mobile device 154, where the augmented-reality environment is accessible by the mobile device 154 through a quick-response code readable by a camera of the mobile device 154.

### 2.1 Computing Device

FIG. 2 is a schematic block diagram of an example device 102 that may be used with one or more embodiments described herein, e.g., as a component of system 100.

Device 102 comprises one or more network interfaces 210 (e.g., wired, wireless, PLC, etc.), at least one processor 220, and a memory 240 interconnected by a system bus 250, as well as a power supply 260 (e.g., battery, plug-in, etc.). Device 102 can further include display device 230 in communication with the processor 220 that displays information to a user.

Network interface(s) 210 include the mechanical, electrical, and signaling circuitry for communicating data over the communication links coupled to a communication network. Network interfaces 210 are configured to transmit and/or receive data using a variety of different communication protocols. As illustrated, the box representing network interfaces 210 is shown for simplicity, and it is appreciated that such interfaces may represent different types of network connections such as wireless and wired (physical) connections. Network interfaces 210 are shown separately from power supply 260, however it is appreciated that the interfaces that support PLC protocols may communicate through power supply 260 and/or may be an integral component coupled to power supply 260.

Memory 240 includes a plurality of storage locations that are addressable by processor 220 and network interfaces 210 for storing software programs and data structures associated with the embodiments described herein. In some embodiments, device 102 may have limited memory or no memory (e.g., no memory for storage other than for programs/processes operating on the device and associated caches). Memory 240 can include instructions executable by the processor 220 that, when executed by the processor 220, cause the processor 220 to implement aspects of the system 100 outlined herein.

Processor 220 comprises hardware elements or logic adapted to execute the software programs (e.g., instructions) and manipulate data structures 245. An operating system 242, portions of which are typically resident in memory 240 and executed by the processor, functionally organizes device 102 by, inter alia, invoking operations in support of software processes and/or services executing on the device. These software processes and/or services may include monoscopic photogrammetry processes/services 290 that implement aspects of the system 100 and associated methods described herein. Note that while monoscopic photogrammetry processes/services 290 is illustrated in centralized memory 240, alternative embodiments provide for the process to be operated within the network interfaces 210, such as a component of a MAC layer, and/or as part of a distributed computing network environment.

It will be apparent to those skilled in the art that other processor and memory types, including various computer-readable media, may be used to store and execute program instructions pertaining to the techniques described herein. Also, while the description illustrates various processes, it is expressly contemplated that various processes may be embodied as modules or engines configured to operate in accordance with the techniques herein (e.g., according to the functionality of a similar process). In this context, the term module and engine may be interchangeable. In general, the term module or engine refers to model or an organization of interrelated software components/functions. Further, while the monoscopic photogrammetry processes/services 290 is shown as a standalone process, those skilled in the art will appreciate that this process may be executed as a routine or module within other processes.

### 2.2 Neural Network

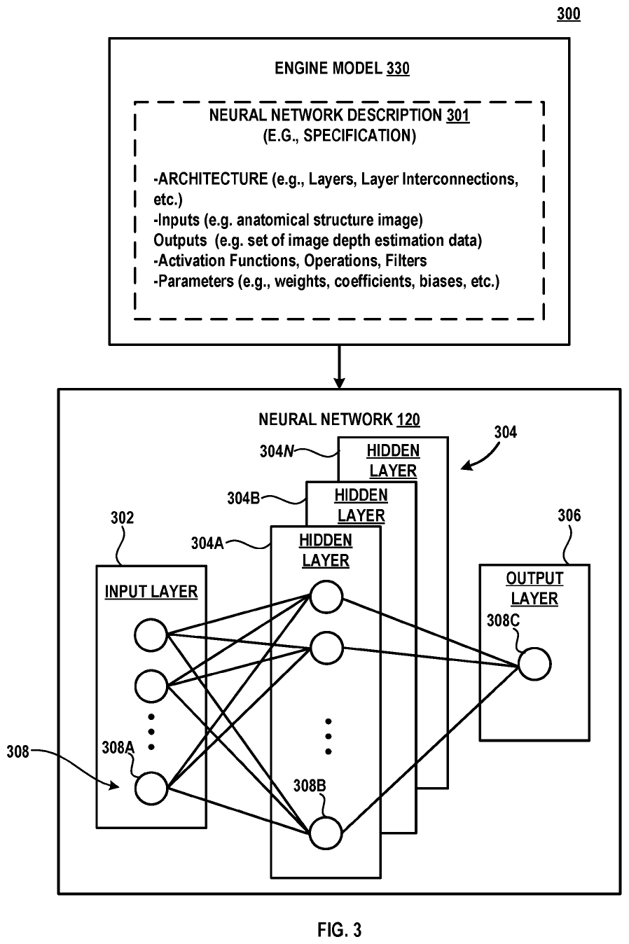

FIG. 3 is a schematic block diagram of an example neural network architecture 300 that may be used with one or more embodiments described herein, e.g., as a component of system 100 shown in FIG. 1A, and particularly as a component of the neural network 120. Possible implementations of the neural network architecture 300 can be used by the system 100 to extract the set of depth estimation data 114 from the 2D anatomical image data 112.

Architecture 300 includes the neural network 120 defined by an example neural network description 301 in an engine model (neural controller) 330. The neural network description 301 can include a full specification of the neural network 120, including the neural network architecture 300. For example, the neural network description 301 can include a description or specification of the architecture 300 of the neural network 120 (e.g., the layers, layer interconnections, number of nodes in each layer, etc.); an input and output description which indicates how the input and output are formed or processed; an indication of the activation functions in the neural network, the operations or filters in the neural network, etc.; neural network parameters such as weights, biases, etc.; and so forth.

The neural network 120 reflects the architecture 300 defined in the neural network description 301. The neural network 120 includes an input layer 302, which receives input data including the 2D anatomical image data 112 corresponding to one or more nodes 308. In one illustrative example, the input layer 302 can include data representing a portion of input media data such as a patch of data or pixels in the 2D anatomical image data 112.

The neural network 120 includes hidden layers 304A through 304N (collectively “304” hereinafter). The hidden layers 304 can include n number of hidden layers, where n is an integer greater than or equal to one. The number of hidden layers can include as many layers as needed for a desired processing outcome and/or rendering intent. The neural network 120 further includes an output layer 306 that provides an output (e.g., the set of depth estimation data 114) resulting from the processing performed by the hidden layers 304. In an illustrative example, the output layer 306 can provide the set of depth estimation data 114 as an image with a color value of each respective pixel corresponding to a “depth” value. As discussed above, the set of depth estimation data 114 can be represented within a comma-separated-variable (.csv) file, a bitmap (.bmp) file, or another suitable file format where each respective pixel of the 2D anatomical image data 112 provided to the input layer 302 is associated with a “depth” value.

The neural network 120 in this example is a multi-layer neural network of interconnected nodes. Each node can represent a piece of information. Information associated with the nodes is shared among the different layers and each layer retains information as information is processed. In some cases, the neural network 120 can include a feed-forward neural network, in which case there are no feedback connections where outputs of the neural network are fed back into itself. In other cases, the neural network 120 can include a recurrent neural network, which can have loops that allow information to be carried across nodes while reading in input.

Information can be exchanged between nodes through node-to-node interconnections between the various layers. Nodes of the input layer 302 can activate a set of nodes in the first hidden layer 304A. For example, as shown, each of the input nodes of the input layer 302 is connected to each of the nodes of the first hidden layer 304A. The nodes of the hidden layer 304A can transform the information of each input node by applying activation functions to the information. The information derived from the transformation can then be passed to and can activate the nodes of the next hidden layer (e.g., 304B), which can perform their own designated functions. Example functions include convolutional, up-sampling, data transformation, pooling, and/or any other suitable functions. The output of the hidden layer (e.g., 304B) can then activate nodes of the next hidden layer (e.g., 304N), and so on. The output of the last hidden layer can activate one or more nodes of the output layer 306, at which point an output is provided. In some cases, while nodes (e.g., nodes 308A, 308B, 308C) in the neural network 120 are shown as having multiple output lines, a node has a single output and all lines shown as being output from a node represent the same output value.

In some cases, each node or interconnection between nodes can have a weight that is a set of parameters derived from training the neural network 120. For example, an interconnection between nodes can represent a piece of information learned about the interconnected nodes. The interconnection can have a numeric weight that can be tuned (e.g., based on a training dataset), allowing the neural network 120 to be adaptive to inputs and able to learn as more data is processed.

The neural network 120 can be pre-trained to process the features from the data in the input layer 302 using the different hidden layers 304 to provide the output through the output layer 306. The neural network 120 can learn to estimate depths from the 2D anatomical image data 112 and can be trained using training data that includes example depth estimations from a training dataset. For instance, training data can be input into the neural network 120, which can be processed by the neural network 120 to generate outputs which can be used to tune one or more aspects of the neural network 120, such as weights, biases, etc.

In some cases, the neural network 120 can adjust weights of nodes using a training process called backpropagation. Backpropagation can include a forward pass, a loss function, a backward pass, and a weight update. The forward pass, loss function, backward pass, and parameter update is performed for one training iteration. The process can be repeated for a certain number of iterations for each set of training media data until the weights of the layers are accurately tuned.

For a first training iteration for the neural network 120, the output can include values that do not give preference to any particular class due to the weights being randomly selected at initialization. For example, if the output is a vector with probabilities that the object includes different product(s) and/or different users, the probability value for each of the different product and/or user may be equal or at least very similar (e.g., for ten possible products or users, each class may have a probability value of 0.1). With the initial weights, the neural network 120 is unable to determine low level features and thus cannot make an accurate determination of what the classification of the object might be. A loss function can be used to analyze errors in the output. Any suitable loss function definition can be used.

The loss (or error) can be high for the first training dataset (e.g., images) since the actual values will be different than the predicted output. The goal of training is to minimize the amount of loss so that the predicted output comports with a target or ideal output. The neural network 120 can perform a backward pass by determining which inputs (weights) most contributed to the loss of the neural network 120 and can adjust the weights so that the loss decreases and is eventually minimized.

A derivative of the loss with respect to the weights can be computed to determine the weights that contributed most to the loss of the neural network 120. After the derivative is computed, a weight update can be performed by updating the weights of the filters. For example, the weights can be updated so that they change in the opposite direction of the gradient. A learning rate can be set to any suitable value, with a high learning rate including larger weight updates and a lower value indicating smaller weight updates.

The neural network 120 can include any suitable neural or deep learning network. One example includes a convolutional neural network (CNN), which includes an input layer and an output layer, with multiple hidden layers between the input and out layers. The hidden layers of a CNN include a series of convolutional, nonlinear, pooling (for downsampling), and fully connected layers. In other examples, the neural network 120 can represent any other neural or deep learning network, such as an autoencoder, a deep belief nets (DBNs), and recurrent neural networks (RNNs), etc.

### 2.3 Training the Neural Network

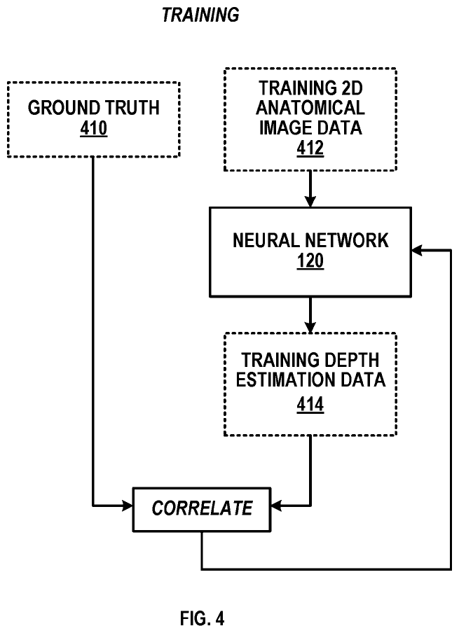

To train the neural network 120, a method includes applying a spatial correlation analysis to the set of (training) depth estimation data produced by the neural network 120 during training with respect to a set of neuronavigation data (e.g., ground truth information 410 that corresponds to training 2D anatomical image data 412 provided as input). In some examples, the neural network 120 can be trained in a multi-stage process involving different types of images and different types of ground truth data such that the neural network 120 can handle different types of data. FIG. 4 shows a training workflow for training the neural network 120. In each stage, a method of training the neural network 120 can include providing training anatomical structure image data 412 to the neural network 120 as input. The neural network 120 can generate a set of training depth estimation data 414 based on the training anatomical structure image data 412. The neural network 120 can be refined, updated, or validated by correlating the set of training depth estimation data 414 with ground truth information 410 including actual empirical depth or linear measurement data.

In the first stage, the training anatomical structure image data 414 can include general anatomical structure images that can be taken from microscopy recordings, etc. The correlation procedure for the first stage can use ground truth information 410 in the form of empirical depth or linear measurement data captured with surgical neuronavigation methods. The first stage can allow the neural network 120 to develop a general framework for generating depth estimation data given an anatomical image. In the second stage, the training anatomical structure image data 412 can include high-resolution cadaveric dissection images. The correlation procedure for the second stage can use ground truth information 410 in the form of empirical depth or linear measurement data captured during cadaveric dissection. The second stage can refine the neural network 120 to provide microscopically accurate depth estimations. In the third stage, the training anatomical structure image data 412 can include different open-access microsurgical images taken from anatomy databases, and the correlation procedure for the third stage can use ground truth information 410 in the form of actual data that accompanies the open-access microsurgical images. The third stage can train the neural network 120 to handle different types of data that might not be uniform or consistent, so that the neural network 120 may be more universal.

### 2.4 Example Implementation

The neural network 120 can be pretrained for depth estimation using images and video data sets, including 3D movies. One such example is the Intel ISL MiDaS (Intel, Santa Clara, CA) model, which has zero cross-data set transfer, meaning performance in data sets not used during the training process. Inputs to the neural network 120 can include 2D anatomical image data 112, and the output can include the set of depth estimation data 114 for the 2D anatomical image data 112. In one aspect, the neural network 120 exports a depth map expressive of the set of depth estimation data 114 for the 2D anatomical image data 112. A depth map is an image where every pixel of the original image is assigned an intensity value according to its location on the Z-axis. From a technical perspective, the neural network 120 (e.g., MiDaS) can compute an inverse depth, allowing training on diverse data (because ground truth data are not always of the same type). For the purposes of this disclosure, inverse depth can be used interchangeably as a surrogate for depth, although it is important to note that the neural network 120 may or may not compute absolute depth. Because every point of the set of depth estimation data 114 can be located in space using specific cartesian coordinates (X, Y, Z), thus creating a 3D point cloud, 3D volumetric reconstructions are possible. Further, the system 100 can include the 3D reconstruction framework 130 including various post-processing modules to generate a 3D anatomical model 116 for 2D anatomical image data 112 based on the set of depth estimation data 114. In one non-limiting example, the 3D reconstruction framework 130 includes Open3D library to create a point cloud from the set of depth estimation data 114, particularly from depth maps obtained by the neural network 120, and further applies additional 3D postprocessing using Meshlab software.

### 2.5 User Interface

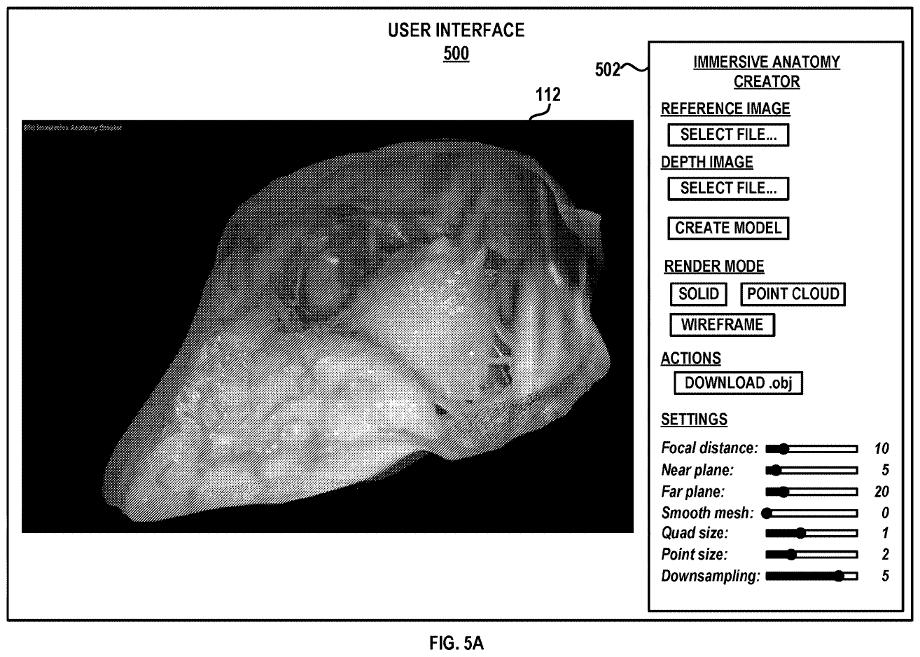

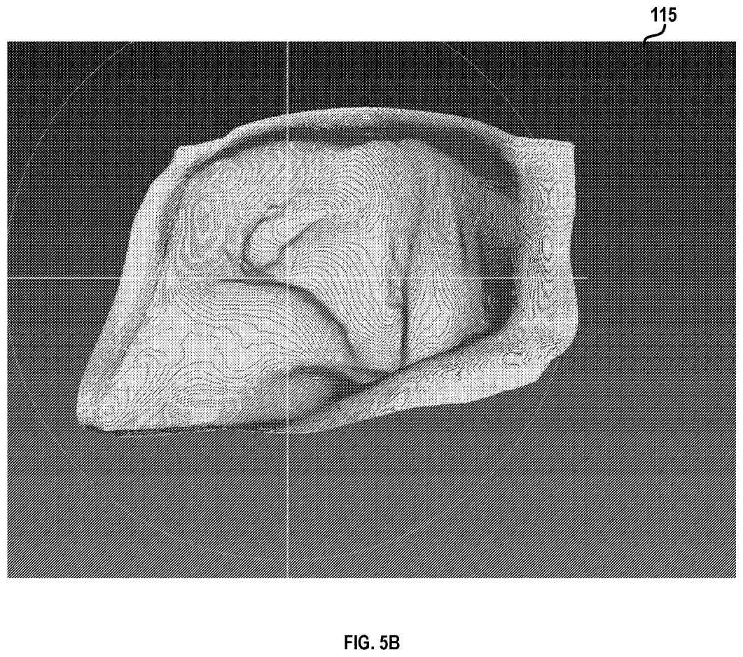

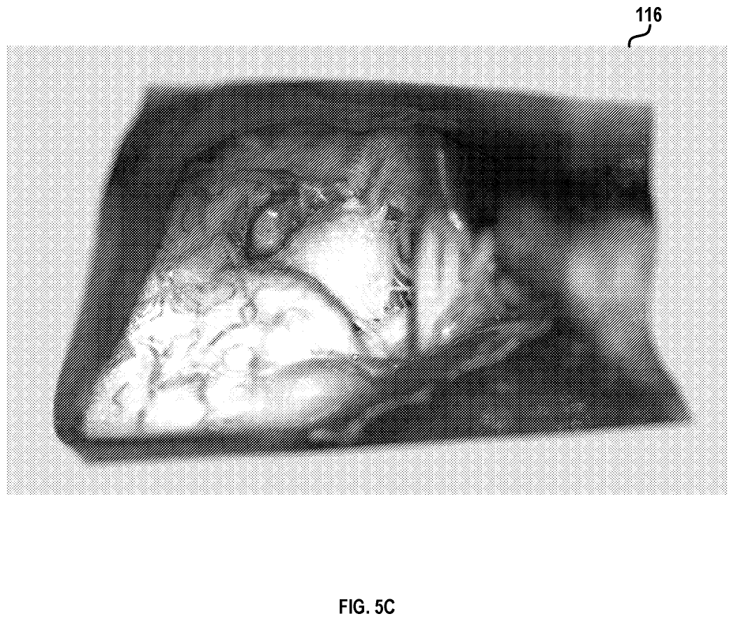

FIGS. 5A-5C show an example user interface 500 of the system 100 and images for generation and viewing of the 3D anatomical model 116. FIG. 5A in particular shows 2D anatomical image data 112 and a menu 502 of the user interface 500 for display at the display device (shown as display device 230 in FIG. 2). In the example shown, the menu 502 can include an option to select and upload a reference image (e.g., the 2D anatomical image data 112) and/or a depth image (e.g., a previously obtained set of depth estimation data 114 for 2D anatomical image data 112). The menu 502 can include a “create model” button that, when interacted with, instructs the system 100 to generate a 3D mesh 115 shown in FIG. 5B and consequently, a final rendering of the 3D anatomical model 116 shown in FIG. 5C. The menu 502 can include a “download.obj” button that enables a computing device of the user to download an object file representative of the 3D anatomical model 116. Other options provided at the menu 502 can include “render mode” settings that display the 3D anatomical model 116 in solid form, in point cloud form, and/or in wireframe form, and can also include other display configurations for illustration. Additional settings can include sliders for focal distance, a near plane, a far plane, a mesh smoothness, a quad size, a point size, and downsampling parameters.

### 2.6 Extended Reality

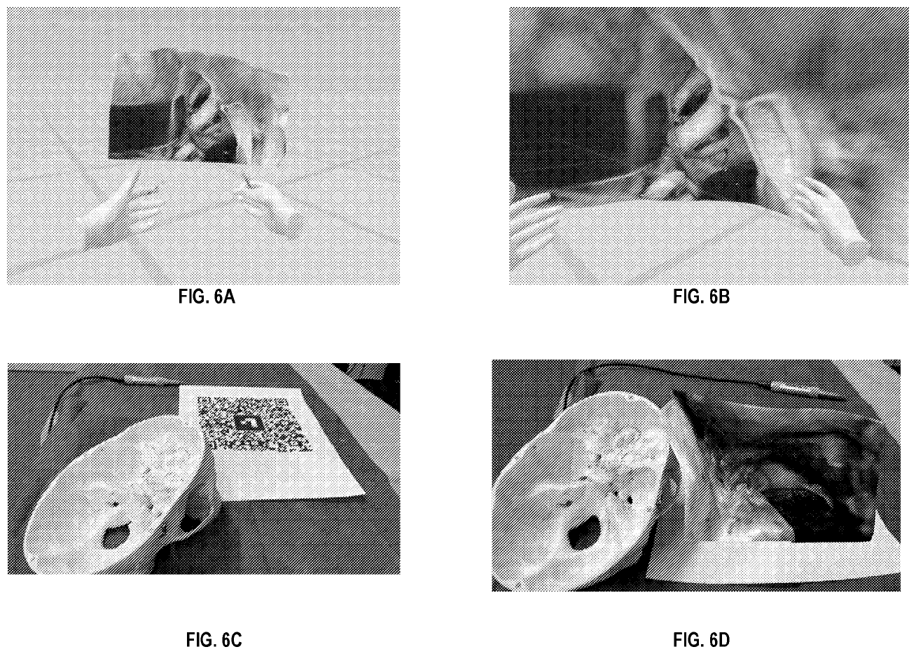

In a further aspect, the system 100 can include the server 104 that hosts the API 140 and is operable for communication with the extended reality device 150 such as virtual reality (VR) display device 152 or mobile device 154. The API 140 can provide instructions executable by a processor associated with the extended reality device 150 to display the final rendering of the 3D anatomical model 116 within an extended reality environment. FIG. 6A extended reality device 150-7D show example environments that can be displayed at the extended reality device 150.

In one example implementation, the server 104 can communicate with the VR display device 152 (FIG. 1A) such as an Oculus Quest headset (Meta Platforms, Inc., Menlo Park, CA). The VR display device 152 can be connected to a computer station for screen casting and video recording. For this implementation, the one or more 3D anatomical models 116 can be provided to the API 140 which can implement a VR collaboration platform such as Spatial (Spatial Dev, New York, NY) to display the final rendering of the 3D anatomical model 116 within a virtual reality environment at the VR display device 152.

In another example, the one or more 3D anatomical models 116 can be provided to the API 140 which can implement an augmented-reality environment for display at the mobile device 154 having a camera such as a tablet (e.g., an iPad Pro 11-inch second generation, Apple, Inc., Cupertino, CA) to display the final rendering of the 3D anatomical model 116 within an augmented reality environment. The API 140 can instruct the mobile device 154 to superimpose the final rendering of the 3D anatomical model 116 over a live feed captured by a camera of the mobile device 154. The augmented-reality environment can be accessible by the mobile device 154 through a quick-response code readable by a camera of the mobile device 154.

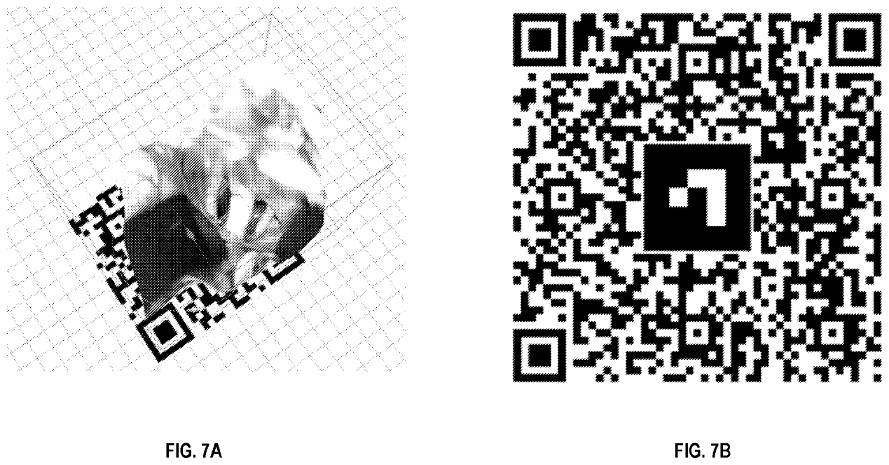

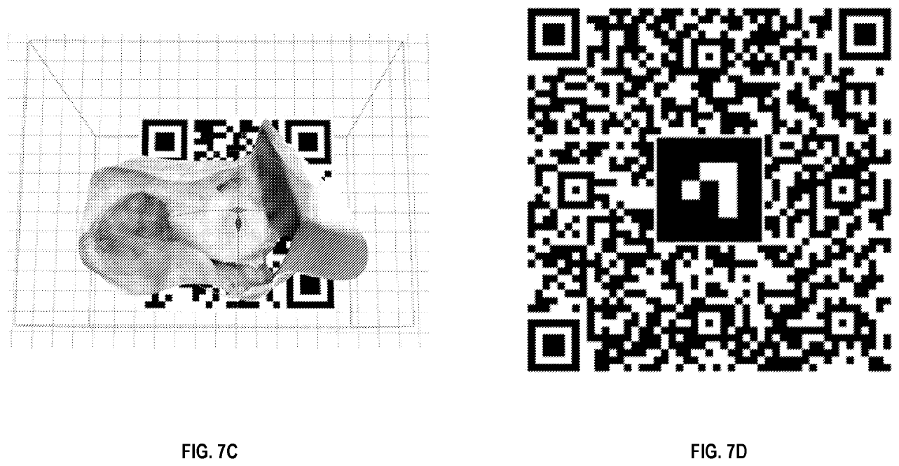

In one such implementation, the augmented-reality environment can be developed using an open-source JavaScript library such as AR.js, with a quick response (QR)-marker-based detection for deployment using the mobile device 154. FIGS. 6A-7D show example images for representation of the one or more 3D anatomical models 116 within the extended reality environment. In particular, FIGS. 6A and 6B illustrate an example display interface at the VR device 152 for display of the one or more 3D anatomical models 116. FIGS. 6C and 6D show example images for display of the one or more 3D anatomical models 116 within the augmented-reality environment; FIG. 6C shows a QR code for scanning by the camera of the mobile device 154 and FIG. 6D shows resultant display of the one or more 3D anatomical models 116 at the mobile device 154 within the augmented-reality environment. FIGS. 7A-7D further show example QR codes and resultant overlay of the one or more 3D anatomical models 116 at the mobile device 154 within the augmented-reality environment.

The 3D methodology of the system 100 produces detailed, realistic 3D anatomical models 116 for display at the VR device 152 (FIGS. 6A and 6B). Head tracking allowed a wearer-visualized perspective for improved recognition of anatomical spatial relations. Using the controllers, the 3D anatomical model 116 displayed at the VR device 152 could be manipulated, including 360° rotation and translation movements. Scale adjustments were possible for higher magnifications. Pial surfaces, arachnoid membranes, cranial nerves, and microvascular structures, including perforators, could be observed in a 3D virtual environment. Multiuser participation in the same VR environment allowed a collaborative educational experience. FIG. 6A shows a visualization of an orbitozygomatic transsylvian approach in virtual reality. FIG. 6B shows the model scale has been increased. The oculomotor nerve, Liliequist membrane, and basilar artery are observed deep in the surgical field.

Augmented reality was assessed with the mobile device 154 (FIGS. 6C and 6D, see also FIGS. 7A-7D). FIG. 6C shows an augmented-reality QR marker is placed on a flat surface for camera detection. FIG. 6D shows an immersive anatomical model of a right extradural approach to the cavernous sinus is displayed. After scanning the QR code, the model was displayed and could be manipulated and rotated in all directions, and the magnification could be increased. Depth perception of anatomical structures could be experienced, albeit in limited fashion, because of knowledge of spatial distribution of the field, even while viewing a screen presenting a 2D image. Augmented reality allowed a side-by-side comparison of virtual models with real models of cranial dissections for improved anatomical learning. The reader can visualize example anatomical models by scanning the QR codes presented in FIGS. 7B and 7D with a mobile device

FIG. 7A shows an anatomical model of the Transsylvian approach incorporated into a QR code augmented-reality experience. FIG. 7B shows an example QR code that can be scanned with mobile devices for visualization of the 3D model of FIG. 7A. FIG. 7C shows an anatomical model of the perilabyrinthine dissection incorporated into a QR code augmented-reality experience. FIG. 7D shows a QR code that can be scanned with mobile devices for visualization of the 3D model of FIG. 7C.

### 3. Materials and Methods

In a first stage of development of one implementation of the system 100, the neural network 120 is first trained to obtain and analyze a set of depth estimation data 114 for the 2D anatomical image data 112. The set of depth estimation data 114 is analyzed through a correlation procedure for correlation with empirical depth or linear measurements obtained through surgical neuronavigation methods to ensure that the neural network 120 can make accurate predictions. In a second stage of development and testing of the system 100, the correlation procedure is applied to high-resolution images of cadaveric brain dissections to correlate the depth estimation data 114 predicted by the neural network 120 with actual measurements. The correlation procedure can also be applied to samples of 2D anatomical image data 112 from an open-access microsurgical anatomy database (e.g., the Rhoton collection). The system 100 enables development of 3D anatomical models 116 using the set of depth estimation data 114 and following verification of correct correlation with respect to actual data. Finally, the 3D anatomical models 116 are exported for display within an extended reality environment for assessment of depth perception and user experience.

### 3.1 Anatomical Study and Photograph Acquisition

To obtain the 2D anatomical image data 112, stepwise dissection of cadaveric specimens can be performed within a specialized neurosurgical laboratory facility using standard microsurgical equipment. In one specific implementation, a Zeiss Pentero microscope (Carl Zeiss Surgical, Inc., Oberkochen, Germany) was used for localized magnification of anatomical structures for fine dissection, and a Medtronic StealthStation S7 (Medtronic, Minneapolis, MN) neuronavigation system was used to define coordinates of relevant anatomical targets.

In one implementation, acquisition of 2D anatomical image data 112 during stepwise dissection was performed using three different cameras: Zeiss Trenion 3D-HD platform with full HD capability, a Canon Eos 5DSr camera with a 50.6-megapixels sensor and a 100-mm lens, and Sony a6000 camera with a 24-megapixels sensor and 50-mm lens complemented with 26-mm rings.

### 3.2 Surgical Technique

Formalin-preserved cadaver heads with artery and vein systems injected with silicone were used for dissection. Using an orbitozygomatic approach, pretemporal transcavernous and the transsylvian corridors were dissected, with exposure of the basilar artery as the deepest anatomical structure within the surgical field. A mastoidectomy with skeletonization of the semicircular canals and fallopian canal was also performed. The jugular bulb was exposed at the infralabyrinthine compartment as the deepest structure of this corridor.

### 3.3 High-Definition Photogrammetry via Monocular Depth Estimation

A query was performed on the Rhoton Collection to obtain 2D anatomical image data 112 for study that meet the following inclusion criteria: clear visualization of a superficial and deep plane, blurring in only one of these planes (blurring in both planes can, in theory, lower accuracy of the depth estimation process), sufficient lighting and brightness, and no images that contain surgical instruments other than retractors (because of potential artifacts).

### 3.4 Correlation Analysis of Depth Estimations and Neuronavigation Measured Depth

To reduce inaccuracy of the set of depth estimation data 114, depth allocations by the neural network 120 are examined through a correlation process to verify consistency of the set of depth estimation data 114 with measurements collected with a neuronavigation system (e.g., a Medtronic StealthStation S7 neuronavigation system). For this process, anatomical regions of interest (ROIs) are selected according to their depth (see Tables 1 and 2). The neuronavigation system is used to measure actual spatial coordinates, and depth is determined for each ROI using Heron's formula, considering the most superficial ROI as the reference value (depth=0 mm). To consider the set of depth estimation data 114 as accurate, an inverse correlation is expected between increasing depth of anatomical structures and mean pixel intensity values measured in the depth map obtained by the neural network 120, where every anatomical structure is allocated a value from a range of 0 (purple) to 255 (yellow), corresponding to the deepest and the most superficial point of the image, respectively.

### 3.5 Statistical Analysis

Stata software version 13.0 (Stata Corp LLC, College Station, TX) is used for statistical analysis. Correlational analysis is performed using Spearman's rho. An alpha value of 50.05 was considered statistically significant.

### 4. Results

One implementation of the system 100 is examined in this section for validation. The depth of each anatomical ROI and corresponding depth estimation (mean pixel intensity value and SD) relative to references points for a pretemporal approach (surface of retractor on frontal lobe) and mastoidectomy (spine of Henle) dissection are presented in Tables 1 and 2, respectively.

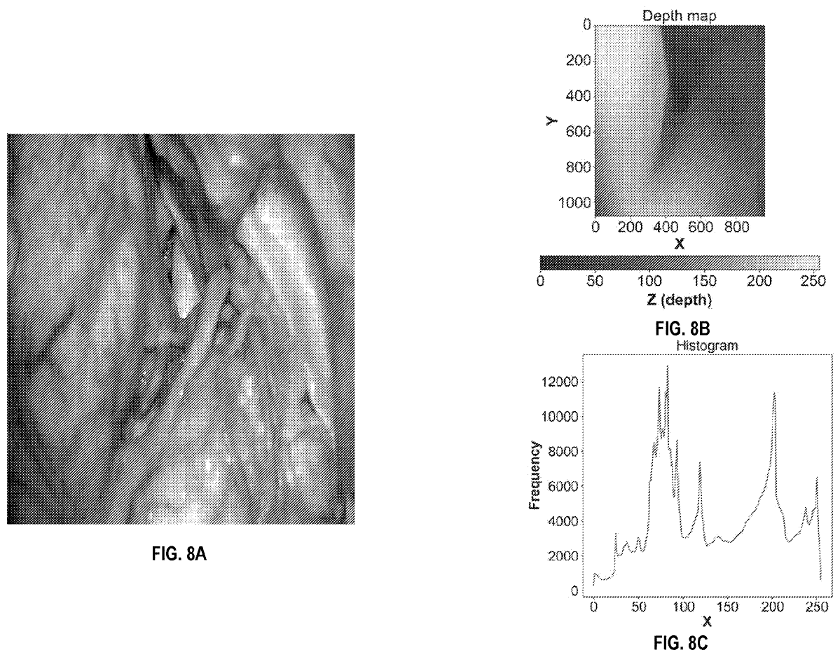

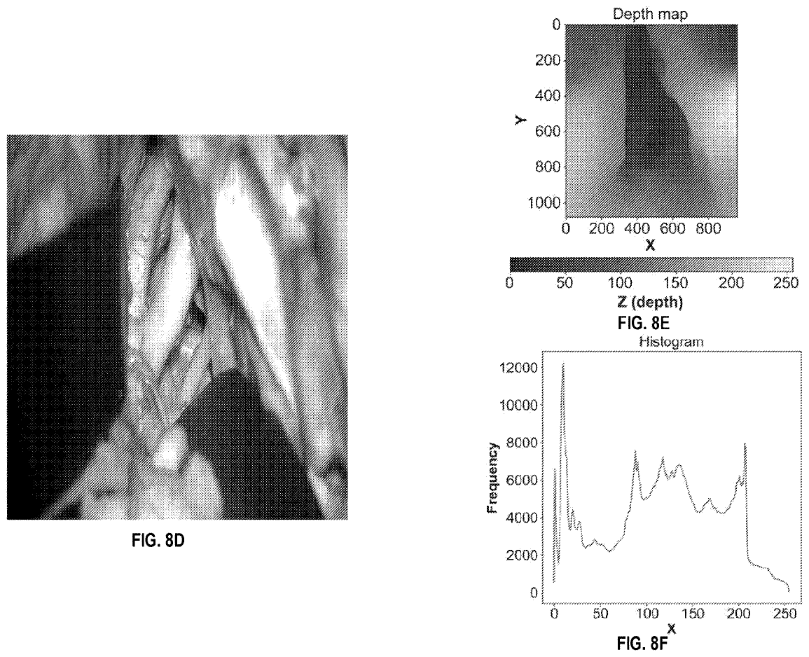

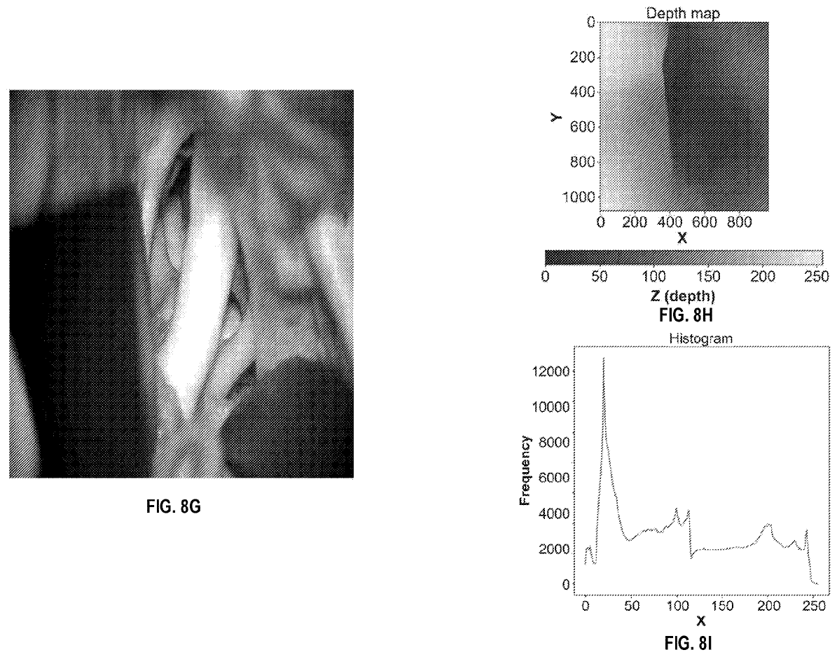

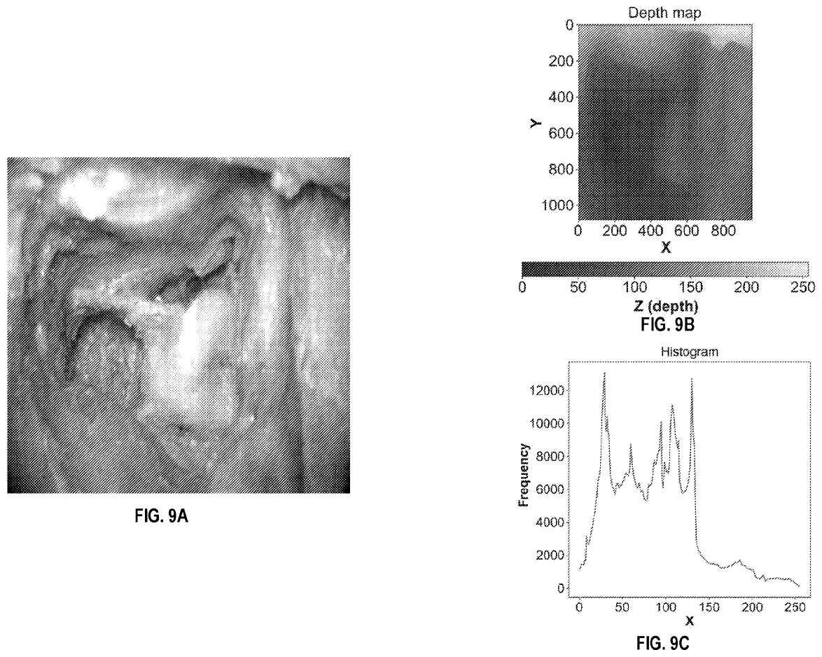

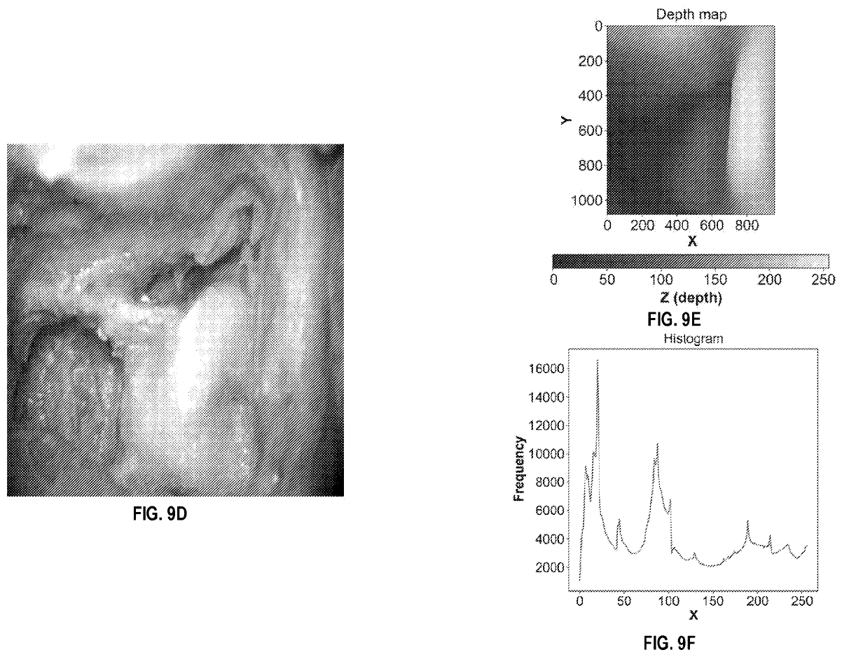

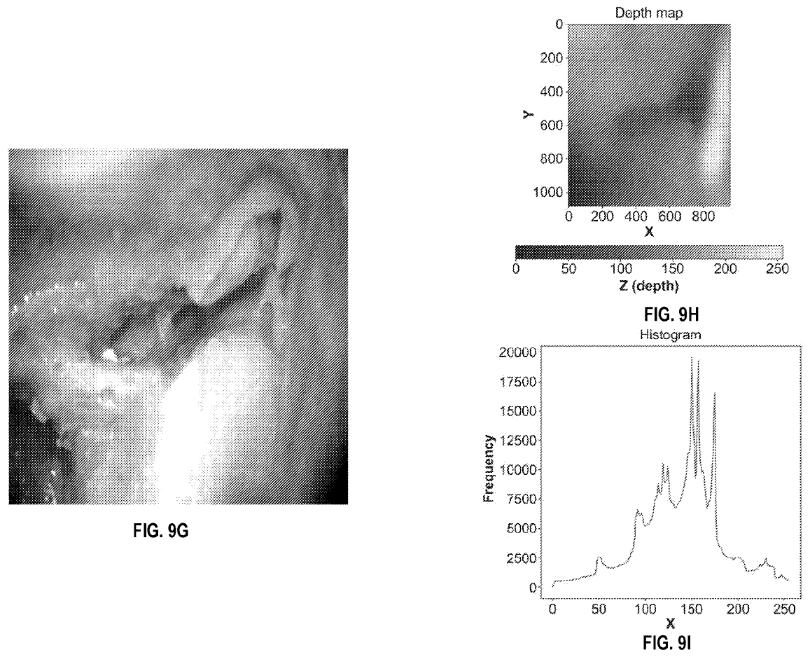

Depth estimations are presented graphically as depth maps and histograms (FIGS. 8A-9I). Analysis of the histograms revealed that pixel distribution changed with different magnifications. In FIGS. 8A-8I (pretemporal dissection), the center of the image corresponded to the deepest region of the surgical field (basilar artery); the histogram revealed an increased pixel allocation toward zero with increasing magnification, a finding consistent with increasing depth allocation by the model.

FIGS. 8A-8I show Transsylvian exposure, right side, at different magnifications. FIG. 9A shows a low-magnification photograph, FIG. 8B shows a depth map of the photograph of FIG. 8A, and FIG. 8C shows a histogram of the photograph of FIG. 8A. FIG. 8D shows an intermediate-magnification photograph, FIG. 8B shows a depth map of the photograph of FIG. 8D, and FIG. 8C shows a histogram of the photograph of FIG. 8D. FIG. 8G shows a high-magnification photograph, FIG. 8H shows a depth map of the photograph of FIG. 8G, and FIG. 8I shows a histogram of the photograph of FIG. 8G, respectively.

FIGS. 9A-9I show a mastoidectomy dissection, the center of the image corresponding to an intermediate region in terms of depth (semicircular canals). The model assigned pixels in the range of 100 to 200 with increasing magnification, meaning the model allocated to an intermediate depth from the most superficial ROI (spine of Henle) and the deepest point (jugular bulb).

FIGS. 9A-9I show Mastoidectomy, left side, at different magnifications. FIG. 9A shows a low-magnification photograph, FIG. 9B shows a depth map of the photograph of FIG. 9A, and FIG. 9C shows a histogram of the photograph of FIG. 9A. FIG. 9D shows an intermediate-magnification photograph, FIG. 9B shows a depth map of the photograph of FIG. 9D, and FIG. 9C shows a histogram of the photograph of FIG. 9D. FIG. 9G shows a high-magnification photograph, FIG. 9H shows a depth map of the photograph of FIG. 9G, and FIG. 9I shows a histogram of the photograph of FIG. 9G, respectively.

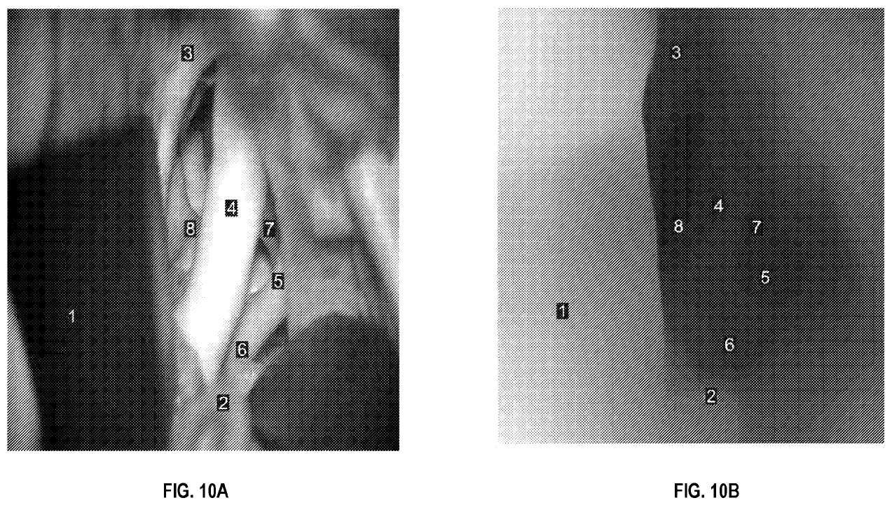

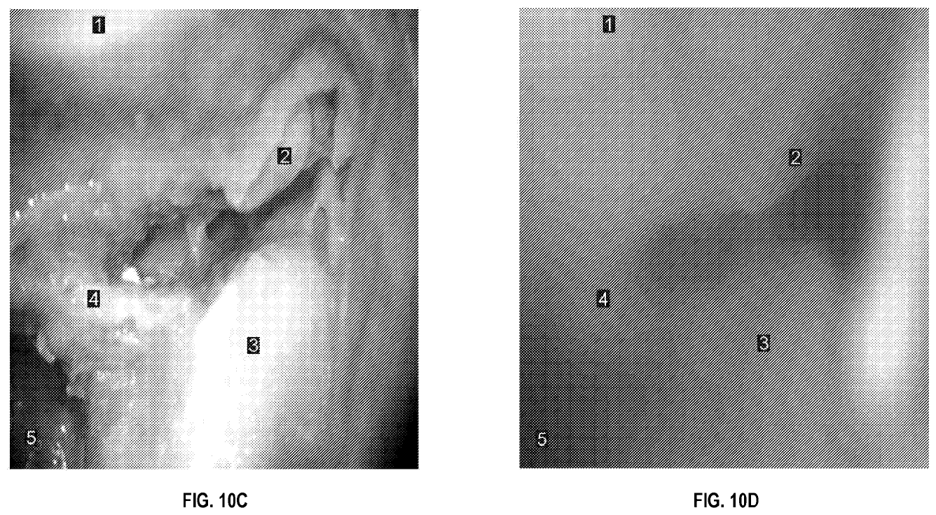

FIGS. 10A-10D show high-magnification images with corresponding depth maps. FIG. 10A shows a transsylvian exposure photograph and FIG. 10B shows a depth map with overlay of anatomical regions of interest used to calculate mean pixel intensity values. Regions of interest: 1=inferior frontal gyrus, 2=middle cerebral artery bifurcation, 3=optic nerve, 4=supraclinoid internal carotid artery, 5=oculomotor porus, 6=oculomotor root entry zone, 7=posterior clinoid process, 8=basilar apex. FIG. 10C shows a mastoidectomy exposure photograph and FIG. 10D shows a depth map with overlay of anatomical regions of interest used to calculate mean pixel intensity values. Regions of interest: 1=spine of Henle, 2=incus, 3=lateral semicircular canal, 4=facial nerve vertical segment, 5=jugular bulb. Quantitative spatial discrimination between the optic nerve (mean±SD pixel intensity: 77.71±0.65), supraclinoid internal carotid artery (69.91±0.33) and the basilar apex (58.98±0.47) is observed in the transsylvian exposure (Table 1, FIGS. 10A and 10B). The incus (128.1±1.0), lateral semicircular canal (131.4±0.7), and vertical segment of the facial nerve (130.6±0.4) were assigned similar mean pixel intensity values with a mastoidectomy exposure, consistent with their known anatomical relations and positions (FIGS. 10C and 10D, Table 2).

TABLE 1
Depth relative to the reference point (surface of retractor on frontal lobe) for each
ROI and the corresponding mean pixel value at all three levels of magnification

| | Measured | | | |
|---|---|---|---|---|
| | depth | Low | Intermediate | High |
| Region of interest | (mm) | magnification | magnification | magnification |
| Inferior frontal gyrus | 0 | 159.05 ± 0.21 | 156.56 ± 0.49 | 149.16 ± 0.82 |
| Superficial sylvian | 10.95 | 140.06 ± 0.81 | 134.57 ± 0.84 | — |
| vein | | | | |
| MCA bifurcation | 41.91 | 106.04 ± 1.61 | 120.17 ± 0.91 | 114.96 ± 1.31 |
| ICA bifurcation | 48.12 | — | 75.57 ± 0.71 | — |
| Optic nerve | 50.78 | 86.78 ± 0.93 | 65.43 ± 0.49 | 77.71 ± 0.65 |
| Supraclinoid ICA | 52.47 | 53.19 ± 0.39 | 63.48 ± 0.50 | 69.91 ± 0.33 |
| Oculomotor nerve | 53.59 | — | 58.71 ± 0.73 | 75.04 ± 0.45 |
| REZ | | | | |
| Oculomotor porus | 55.51 | — | 63.09 ± 4.42 | 68.12 ± 0.59 |
| Posterior clinoid | 57.76 | — | 61.10 ± 0.53 | 65.89 ± 0.55 |
| Basilar apex | 61.39 | — | 53.05 ± 0.21 | 58.98 ± 0.47 |

With respect to Table 1, data are shown as mean pixel intensity±standard deviation unless otherwise noted. Abbreviations: ICA, internal carotid artery; MCA, middle cerebral artery; REZ, root entry zone; ROI, region of interest.

TABLE 2
Depth relative to the reference point (spine of Henle) for each ROI and
the corresponding mean pixel value at all three levels of magnification

| | Measured | | | |
|---|---|---|---|---|
| | depth | Low | Intermediate | High |
| Region of interest | (mm) | magnification | magnification | magnification |
| Spine of Henle | 0 | 139.8 ± 1.1 | 154.5 ± 0.9 | 155.1 ± 0.7 |
| Incus | 26.2 | 99.7 ± 1.1 | 115.9 ± 1.0 | 128.1 ± 1.0 |
| Lateral semicircular | 27.9 | 114.5 ± 0.7 | 118.5 ± 0.5 | 131.4 ± 0.7 |
| canal | | | | |
| Facial nerve, vertical | 29.0 | 77.5 ± 0.5 | 75.7 ± 0.7 | 130.6 ± 0.4 |
| segment | | | | |
| Jugular bulb | 36.0 | 77.1 ± 0.32 | 69.2 ± 0.4 | 67.5 ± 2.2 |

With respect to Table 2, data are shown as mean pixel intensity±standard deviation unless otherwise noted.

Correlation analysis between pixel intensity values and measured depth revealed statistically significant inverse correlations. For the pretemporal approach dissection, the Spearman's rho was −1.0 for the low-magnification image (p<0.0001), −0.98 for intermediate magnification (p<0.0001), and −0.97 for high magnification (p<0.0001). For the posterior temporal bone dissection, the Spearman's rho was −0.90 (p=0.04) for each magnification studied.

### 4.1 Anatomical 3D Construction

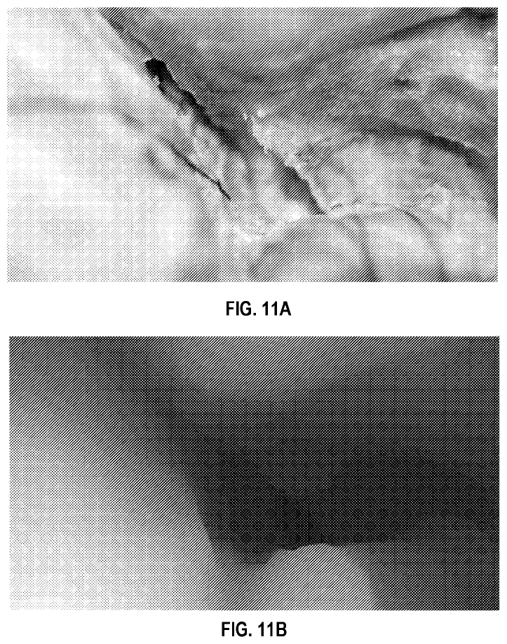

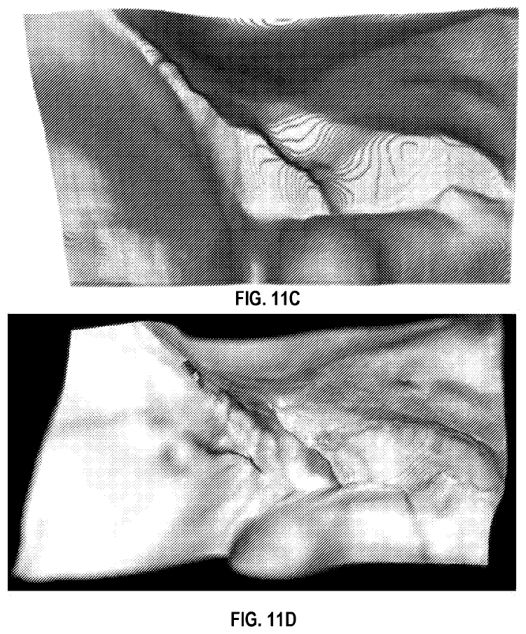

The 3D methodology process applied by the system 100 is demonstrated in FIGS. 11A-11D.

FIGS. 11A-11D show a 3D reconstruction procedure. FIG. 11A shows a microsurgical image provided as input to the neural network 120, FIG. 11B shows a depth map generated by the neural network 120, FIG. 11C shows a mesh surface reconstruction generated by the 3D reconstruction framework 130, and FIG. 11D shows a final immersive anatomical model generated by the 3D reconstruction framework 130 from the mesh surface reconstruction.

Access to example 3D models is available online: orbytozigomatic pretemporal approach, perilabyrinthine approach, and extradural approach to the cavernous sinus.

### 4.2 Discussion

The principle of “a picture is worth a thousand words” is true in anatomy education: a high-quality microneurosurgical image can provide greater understanding than long paragraphs of descriptive anatomical observations and measurements. In this study, monoscopic photogrammetry using machine learning produced 3D reconstructions of microsurgical images. This allowed creation of navigable immersive anatomy experiences that were accurate in terms of spatial representation even with high magnification, enhancing the understanding of complex anatomical relationships.

### 4.3 3D Extended Reality and Neurosurgery Education

Mastery of anatomical orientation and visuospatial skills are necessary for a neurosurgeon to perform microsurgery with comfort and confidence and should be a fundamental part of any neurosurgery training program. Stereopsis, the optical-neurological phenomenon producing depth perception, is fundamental to a neurosurgeon's understanding of anatomical relationships in small spaces. The cadaver dissection laboratory is the ideal environment for gaining this type of mastery, yet the availability of this training has many well-known limitations. The COVID-19 pandemic has further accelerated the trend toward extended reality, which can be used remotely. In fact, remote learning has become the mainstay of education during the pandemic.

Extended reality simulators for neurosurgery training have been in development for more than 20 years, and their use has increased in many centers worldwide. Using DICOM data from neuroimaging studies, 3D rendering of anatomical models is possible with accurate spatial localization of structures. However, from a microsurgical training perspective, these 3D renderings may be cartoonish or artificial and often fail to emulate essential features such as textures, colors, and lighting of the exposed neurovascular structures that must be identified when performing complex cranial approaches.

### 4.4 Photogrammetry: Traditional Versus Present System

Traditional photogrammetry requires acquisition of multiple images for 3D reconstruction. A pioneer experience using a robotic microscope for stereoscopic photogrammetry performed over 20 years ago was one of the most realistic simulators of neurosurgical approaches at the time but required specialized and expensive equipment. Currently, photogrammetry is commonly performed using software that superimposes multiple images of a subject and, through a triangulation process, creates a volumetric surface reconstruction. Immersive anatomy experiences of cranial approaches developed with photogrammetry have been reported by Rodriguez Rubio et al. and others. Similarly, Qlone, an app that performs 360° photogrammetry using mobile devices, has been used to create macroscopic and sectional 3D models of the brain and brainstem.

However, a caveat of photogrammetry is that it is difficult to create 3D models that accurately represent a microsurgical perspective. Some important neuroanatomical structures are only visible from very restricted angles, and mobile devices do not provide the same field of view as a microscope. Because our goal is to faithfully preserve the approach perspective, the use of only one image as input for 3D reconstruction is consistent. By using a neural network, we captured the surgical perspective of these narrow corridors and developed 3D models using an image from the microscope.

The present disclosure establishes that machine learning can deliver plausible anatomical depth estimations for specific sets of ROIs using only one image as input. This does not mean that depth allocations will be always accurate; it is necessary to perform a cross-check against neuronavigation or DICOM (Digital Imaging and Communications in Medicine) data to verify the accuracy of estimations. Through observation and validation of the system 100, however, that in the case of Rhoton Collection models, even without ground truth measurements available, overall 3D reconstruction by the system 100 is possible and anatomically consistent. The relationship of one anatomical structure to another made intuitive sense and was realistic overall.

### 4.5 Example of Augmenting the Rhoton Collection and Beyond

The system 100 can be applied to existing surgical image compendiums to enhance education. In one example, the Rhoton Collection, with its lectures and photographs of anatomical dissections using 3D stereoscopy that facilitate understanding of spatial relationships between complex structures, was used as a prime example of a compendium of images to which the system 100 could offer added value, allowing conversion of classic anatomical dissections into conveniently accessible, viewable, immersive, 3D, realistic environments. Further development of anatomy simulators based on other surgical dissection repositories and real-time surgery is an exciting possibility with this technology. Collaborative VR anatomical training with digital performance metrics and artificial intelligence integration for surgical performance classification could augment cadaver dissection in the future, even remotely.

### 4.6 Conclusions

Anatomical depth estimations calculated by pretrained models are relative estimations and are subject to differences with absolute spatial measurement. Further training and fine-tuning of the neural network 120 (and potential inclusion of additional or alternative neural networks) with a large data set of microsurgical images with “ground truth” information should increase the accuracy of estimations. In addition, individual coordinates co-registered from the neuronavigation system might not reflect in vivo reality due to the nature of cadaveric specimens and the natural phenomenon of brain shifting.

The system 100 implements machine learning principles for depth estimation of anatomical structures in microsurgical approaches using only one image as input. The methodologies discussed herein are inherently different from other forms of photogrammetry, which require multiple images, different angles, and prior knowledge of the structures. There appears to be no preceding studies that introduce 3D anatomical rendering of a single photo (monoscopic photogrammetry) and apply the 3D anatomical rendering to the field of microneurosurgery anatomy. The system 100 and associated methodologies allow creation of immersive, realistic, anatomical models useful for augmenting neuroanatomy education. Such a process could be especially convenient where either the availability of cadaveric specimens is limited or microsurgical dissection laboratories are absent, or merely when enhanced comprehension of the spatial relation of anatomical structures is desired.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. A system, comprising:
a processor in communication with a memory, the memory including instructions executable by the processor to:
access a set of two-dimensional anatomical image data;
generate a set of depth estimation data based on the set of two-dimensional anatomical image data by a monoscopic photogrammetry procedure;
generate a three-dimensional mesh of a three-dimensional anatomical model using the set of depth estimation data; and
generate a final rendering of the three-dimensional anatomical model using the set of two-dimensional anatomical image data and the three-dimensional mesh.

2. The system of claim 1, the memory including instructions further executable by the processor to:
generate the set of depth estimation data by a neural network, the neural network accepting the set of two-dimensional anatomical image data as input and being trained to output the set of depth estimation data that correlates with the set of two-dimensional anatomical image data.

3. The system of claim 2, the memory including instructions further executable by the processor to:
compute an inverse depth value for each two-dimensional coordinate pair of a plurality of two-dimensional coordinate pairs of the set of two-dimensional anatomical image data; and
generate a three-dimensional point cloud of the set of depth estimation data by combination of each two-dimensional coordinate pair and the inverse depth value for the plurality of two-dimensional coordinate pairs of the set of two-dimensional anatomical image data.

4. The system of claim 3, the memory including instructions further executable by the processor to:
convert the three-dimensional point cloud to the three-dimensional mesh of the three-dimensional anatomical model.

5. The system of claim 1, the memory further including instructions executable by the processor to:
apply a spatial correlation analysis to the set of depth estimation data with respect to a set of neuronavigation data.

6. The system of claim 5, the set of depth estimation data being generated by a neural network, the neural network being trained by correlating a set of training depth estimation data generated by the neural network with empirical depth and/or linear measurements obtained through surgical neuronavigation and/or cadaveric dissection.

7. The system of claim 5, the set of depth estimation data being generated by a neural network, the neural network being trained by correlating a set of training two-dimensional anatomical image data provided as input to the neural network with empirical depth and/or linear measurements obtained through surgical neuronavigation and/or cadaveric dissection.

8. The system of claim 1, further comprising:
an application programming interface hosted at a server operable for communication with a virtual reality display device, the application programming interface providing instructions executable by a processor associated with the virtual reality display device to display the final rendering of the three-dimensional anatomical model within a virtual reality environment.

9. The system of claim 1, further comprising:
an application programming interface hosted at a server operable for communication with a mobile device, the application programming interface providing instructions executable by a processor of the mobile device to display the final rendering of the three-dimensional anatomical model within an augmented-reality environment.

10. The system of claim 9, the application programming interface providing instructions executable by the processor of the mobile device to:
superimpose the final rendering of the three-dimensional anatomical model over a live feed captured by a camera of the mobile device.

11. The system of claim 9, the augmented-reality environment being accessible by the mobile device through a quick-response code readable by a camera of the mobile device.

12. A method, comprising:
accessing a set of two-dimensional anatomical image data;
generating a set of depth estimation data based on the set of two-dimensional anatomical image data by a monoscopic photogrammetry procedure;
generating a three-dimensional mesh of a three-dimensional anatomical model using the set of depth estimation data; and
generating a final rendering of the three-dimensional anatomical model using the set of two-dimensional anatomical image data and the three-dimensional mesh.

13. The method of claim 12, further comprising:
generating the set of depth estimation data by a neural network, the neural network accepting the set of two-dimensional anatomical image data as input and being trained to output the set of depth estimation data that correlates with the set of two-dimensional anatomical image data.

14. The method of claim 13, further comprising:
computing an inverse depth value for each two-dimensional coordinate pair of a plurality of two-dimensional coordinate pairs of the set of two-dimensional anatomical image data; and
generating a three-dimensional point cloud of the set of depth estimation data by combination of each two-dimensional coordinate pair and the inverse depth value for the plurality of two-dimensional coordinate pairs of the set of two-dimensional anatomical image data.

15. The method of claim 14, further comprising:
converting the three-dimensional point cloud to the three-dimensional mesh of the three-dimensional anatomical model.

16. The method of claim 12, further comprising:
applying a spatial correlation analysis to the set of depth estimation data with respect to a set of neuronavigation data.

17. The method of claim 16, the set of depth estimation data being generated by a neural network, the neural network being trained by correlating a set of training depth estimation data generated by the neural network with empirical depth and/or linear measurements obtained through surgical neuronavigation and/or cadaveric dissection.

18. The method of claim 16, the set of depth estimation data being generated by a neural network, the neural network being trained by correlating a set of training two-dimensional anatomical image data provided as input to the neural network with empirical depth and/or linear measurements obtained through surgical neuronavigation and/or cadaveric dissection.

19. The method of claim 12, further comprising:
communicating the final rendering of the three-dimensional anatomical model to a server hosting an application programming interface operable for communication with a mobile device, the application programming interface providing instructions executable by a processor of the mobile device to display the final rendering of the three-dimensional anatomical model within an augmented-reality environment.

20. The method of claim 12, further comprising:
communicating the final rendering of the three-dimensional anatomical model to a server hosting an application programming interface operable for communication with a virtual reality display device, the application programming interface providing instructions executable by a processor associated with the virtual reality display device to display the final rendering of the three-dimensional anatomical model within a virtual reality environment.
